(19) United States
(12) Patent Application Publication
(10) Pub. No.: US 2006/0173498 A1
(43) Pub. Date: Aug. 3, 2006

(54) Communication between an external defibrillator and an implantable medical device

(76) Inventors: Isabelle Banville, Sammamish, WA (US); Fred William Chapman, Newcastle, WA (US); Robert G. Walker, Bothell, WA (US); Joseph Leo Sullivan, Kirkland, WA (US); Richard C. Nova, Kirkland, WA (US)

Correspondence Address: SHUMAKER & SIEFFERT, P. A., 8425 SEASONS PARKWAY, SUITE 105, ST. PAUL, MN, 55125, US

(21) Appl. No.: 11/047,482

(22) Filed: Jan. 31, 2005

(51) Int. Cl. A61N 1/39

(52) U.S. Cl. 607/5

(57) ABSTRACT

An external defibrillator used to treat a patient wirelessly communicates with an implantable medical device (IMD) implanted in the patient. In some embodiments, a telemetry head may be removably coupled to the external defibrillator to facilitate communication with the IMD. The defibrillator may receive information, such as patient, device, physiological, and treatment information, from the IMD. The defibrillator may use some or all of this information during treatment of the patient. For example, the defibrillator may prompt a user, or modify programmed user prompts based on the information. The defibrillator may deliver therapy based on the information. The information may include real-time values of physiological parameters monitored by the IMD, which may be displayed by the external defibrillator. The external defibrillator may control delivery of therapy by the IMD. The defibrillator may collect medical event information during treatment of the patient, and store the medical event information in the IMD.

### TECHNICAL FIELD

The invention relates to medical devices and, more particularly, to medical device communication.

### BACKGROUND

An external defibrillator delivers energy to a heart of a patient via electrodes placed upon the patient's chest. Often, external defibrillators are used to deliver energy in the form of a defibrillation pulse to a heart that is undergoing ventricular fibrillation and has lost its ability to contract. Ventricular fibrillation is particularly life threatening because activity within the ventricles of the heart is so uncoordinated that virtually no pumping of blood takes place. If untreated, the patient whose heart is undergoing fibrillation may die within a matter of minutes.

An electrical pulse delivered to a fibrillating heart may depolarize the heart and cause it to reestablish a normal sinus rhythm. In some cases, the patient may need multiple pulses, and the external defibrillator may deliver different quantities of energy with each defibrillation pulse. Further, the defibrillator may provide additional or alternative therapies to the patient, such as cardioversion or pacing therapy. As examples, the external defibrillator may be an automated external defibrillator (AED) used by a first responder or bystander to treat the patient, or a more fully-featured defibrillator/monitor used by paramedics.

In some cases, a patient treated by an external defibrillator may have previously received an implantable medical device (IMD), such as an implantable pacemaker or pacemaker with cardioversion and/or defibrillation capabilities. IMDs typically include telemetry circuitry and an antenna for wireless communication with other devices, such as external programming devices. An example of a programming device that is capable of communicating with IMDs is the Medtronic Model 9790 programmer, commercially available from Medtronic, Inc., and described in U.S. Pat. Nos. 5,345,362 and 5,527,348 to Winkler et al. As another example, U.S. Pat. No. 6,477,424 to Thompson et al. describes an IMD communication system that includes a module interface apparatus that facilitates communication between an IMD and a medical information management system. A commercial embodiment of such an IMD communication is the Carelink® network provided by Medtronic, Inc. However, unlike these examples, conventional external defibrillators are unable to communicate with IMDs.

### SUMMARY

In general, the invention is directed to techniques for providing therapy to a patient and managing medical information through communication between an external defibrillator used to treat the patient and an implantable medical device (IMD) implanted within the patient. As examples, the external defibrillator may receive information from the IMD, prompt a user based on information received from the IMD, deliver therapy based on information received from the IMD, control delivery of therapy by the IMD, and store information within the IMD. Through communication with an IMD according to the invention, an external defibrillator may provide more effective treatment to a patient in which the IMD is implanted, and may more effectively manage medical information than is possible with conventional defibrillators that are incapable of communicating with IMDs.

An external defibrillator wirelessly communicates with an IMD, i.e., without being coupled to the IMD by a wire or other electrical conductor. In some embodiments, the external defibrillator wirelessly communicates with an IMD via telemetry circuitry of the IMD, which the IMD may also use to communicate with dedicated programming devices. The defibrillation may communicate with the IMD via a radio-frequency (RF) medium, e.g., via RF telemetry. In some embodiments, a telemetry head may be removably coupled to the external defibrillator to facilitate communication with the IMD. The telemetry head may include an antenna to facility RF communication, and may also include telemetry circuitry.

The external defibrillator may receive information, such as patient, device, physiological, and treatment information, from the IMD. For example, the information may include patient treatment alerts, which may indicate allergies of the patient, medications taken by the patient, or a do not resuscitate (DNR) order for the patient. Device information received from the IMD may include an implant location of the IMD. Further, the information may include real-time values of physiological parameters monitored by the IMD, such as a real-time electrocardiogram (ECG).

In some embodiments, the IMD may determine the time at which a medical emergency involving the patient began, e.g., when the patient first experienced a fibrillation or sudden cardiac arrest (SCA). In embodiments in which the IMD is a cardiac pacemaker, for example, the IMD may detect onset of fibrillation or SCA through analysis of an electrocardiogram of the patient. In some embodiments, the IMD may include one or more DC accelerometers, mercury switches, or gyroscopes to detect patient posture, and may detect a collapse associated with a medical emergency, such as fibrillation or SCA. The external defibrillator may receive information indicating the time of onset of the medical emergency from the IMD.

The external defibrillator may provide information received from the IMD to a user in the form of prompts. In some embodiments, the external defibrillator may modify programmed user prompts based on the information received from the IMD. For example, the external defibrillator may modify a prompt directing a user to place electrodes at a location on the patient based on implant location information received from the IMD. Further, the external defibrillator may display information received from the IMD, such as real-time values of physiological parameters monitored by the IMD. As an example, the external defibrillator may display a real-time ECG received from the IMD. Additionally, the external defibrillator may display a time of onset of the medical emergency received from the IMD.

In some embodiments, the external defibrillator may deliver therapy to the patient based on information received from the IMD implanted within the patient. For example, the external defibrillator may select an energy level for a defibrillation pulse to be delivered to the patient based on an energy level of a defibrillation pulse previously delivered to the patient by the IMD. As another example, the external defibrillator may analyze an ECG received from the IMD to determine whether to deliver a defibrillation pulse to the patient. Further, the external defibrillator may prompt a user to perform cardiopulmonary resuscitation (CPR) rather than recommending delivery or delivering defibrillation pulses to the patient, based on a time of onset of the medical emergency received from the IMD.

In some embodiments, the external defibrillator may control delivery of therapy by the IMD. For example, the external defibrillator may change a therapy delivery mode of the IMD, such as a pacing mode in embodiments in which the IMD is a cardiac pacemaker. Further, the external defibrillator may coordinate delivery of therapy to the patient with the IMD. For example, the external defibrillator may control the IMD to deliver a defibrillation pulse simultaneously with, or with some other temporal relationship to, delivery of a defibrillation pulse by the external defibrillator. As another example, the external defibrillator and IMD may cooperate to provide post extra-systolic potentiation (PESP) pacing therapy.

The external defibrillator may collect medical event information during treatment of the patient, which may be used by a user of the external defibrillator to prepare a report documenting the treatment of the patient with the defibrillator. The external defibrillator may store information received from the IMD as part of the medical event information. Further, the external defibrillator may store the medical event information within the IMD for later retrieval by a physician or the like using a programming device, or later transmission from the IMD to a computing device, computing network, or other data repository, which may be located at, for example, a hospital.

In one embodiment, the invention is directed to an external defibrillator comprising wireless communication circuitry to wirelessly communicate with an implantable medical device implanted within a patient, therapy delivery circuitry, and a processor. The processor receives information from the implantable medical device via the wireless communication circuitry, and controls the therapy delivery circuitry to deliver therapy to the patient based on the received information.

In another embodiment, the invention is directed to an external defibrillator comprising wireless communication circuitry to wirelessly communicate with an implantable medical device implanted within a patient and a processor. The processor receives real-time values of a physiological parameter of the patient from the implantable medical device via the wireless communication circuitry.

In another embodiment, the invention is directed to a method comprising wirelessly communicating with an implantable medical device implanted within a patient via an external defibrillator, and receiving real-time values of a physiological parameter of the patient from the implantable medical device at the external defibrillator via the wireless communication.

In another embodiment, the invention is directed to an external defibrillator comprising wireless communication circuitry to wirelessly communicate with an implantable medical device implanted within a patient, a user interface, and a processor. The processor receives information from the implantable medical device via the wireless communication circuitry, and prompts a user of the external defibrillator via the user interface based on the information.

In another embodiment, the invention is directed to a method comprising wirelessly communicating with an implantable medical device implanted within a patient via an external defibrillator, receiving information from the implantable medical device at the external defibrillator via the wireless communication, and prompting a user of the external defibrillator based on the received information.

The invention may provide advantages. For example, through communication with an IMD, an external defibrillator according to the invention may be able to provide types of information to a user that the user may not have been otherwise able to obtain, such as a time of onset of the medical emergency. Further, the external defibrillator may have access to sensor data for physiological parameters from the IMD that the external defibrillator could not itself have obtained, or at a higher quality than the external defibrillator could have itself obtained.

In some embodiments, an external defibrillator may be able to make therapy decisions more accurately based on information, such as physiological parameter values or time of onset of the medical emergency, received from the IMD than would be possible in the absence of such information. Additionally, an external defibrillator that receives information regarding therapies delivered by an IMD from the IMD may be able to avoid delivering redundant therapies to patient. Further, through communication with an IMD, an external defibrillator may be able to deliver coordinated therapies with the IMD that may be more effective than therapies delivered by the IMD or external defibrillator alone, or could not have been delivered by the IMD or external defibrillator alone. The external defibrillator and IMD may communicate and operate synergistically to provide a patient in whom the IMD is implanted the most suitable treatment available from either device individually, or from both devices acting in a coordinated manner.

Further, an external defibrillator that receives information from an IMD may allow a user to compile more complete reports of the treatment of the patient. Additionally, an external defibrillator that stores medical event information collected during the treatment of the patient within the IMD may allow a caregiver or hospital that retrieves the information from the IMD to have a more complete record of the treatment of the patient with the external defibrillator. Using the medical event information retrieved from the IMD, the caregiver or hospital may be able to give the patient more effective treatment.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example system that includes an external defibrillator communicating with an implantable medical device implanted within a patient.

FIG. 2 is a block diagram further illustrating the implantable medical device of FIG. 1.

FIG. 3 is a block diagram further illustrating the external defibrillator of FIG. 1.

FIG. 4 is a block diagram illustrating an example telemetry head that may be used to enable communication between an external defibrillator and an implantable medical device.

FIG. 5 is a flow diagram illustrating an example method that may be employed by an external defibrillator that communicates with an implantable medical device during treatment of a patient.

### DETAILED DESCRIPTION

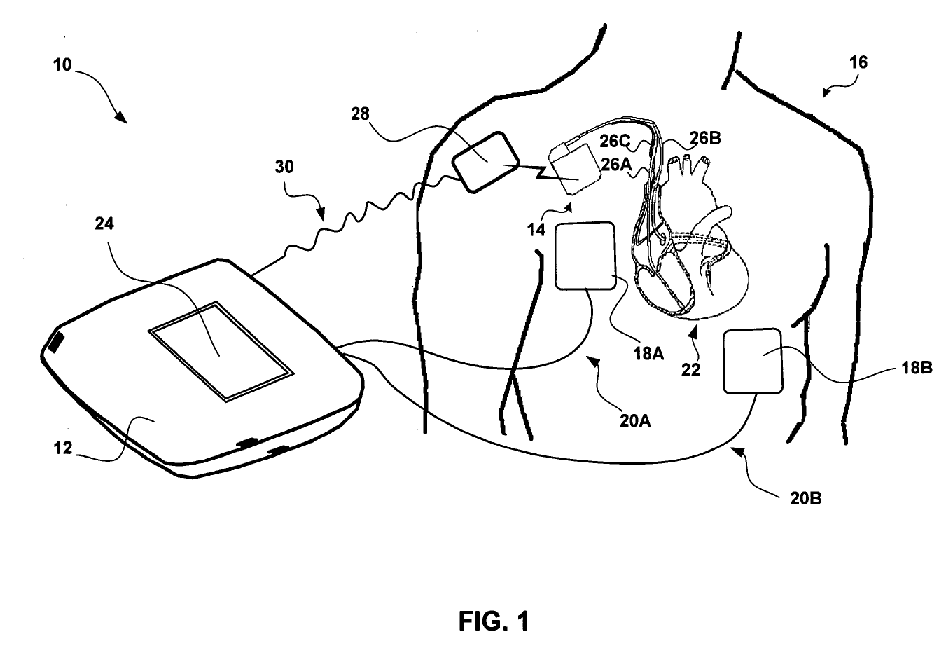

FIG. 1 is a conceptual diagram illustrating an example system 10 that includes an external defibrillator 12 wirelessly communicating with an implantable medical device (IMD) 14 implanted within a patient 16. As examples, external defibrillator 12 may receive information from IMD 14, prompt a user based on information received from IMD 14, deliver therapy based on information received from IMD 14, control delivery of therapy by IMD 14, and store information within IMD 14. Through communication with IMD 14, external defibrillator 12 may provide more effective treatment of patient 16 and medical information management than is possible with conventional external defibrillators that are incapable of communicating with IMDs.

External defibrillator 12 may detect IMD 14 and initiate communication with the IMD when external defibrillator 12 is brought into proximity with patient 16. For example, external defibrillator 12 may be brought to patient 16 in response to a medical emergency involving the patient, such as a ventricular fibrillation (VF) or sudden cardiac arrest (SCA) experienced by the patient. External defibrillator 12 may be, for example, an automated external defibrillator (AED), or a more fully featured external defibrillator/monitor.

In the illustrated example, external defibrillator 12 is coupled to two electrodes 18A and 18B (collectively “electrodes 18”) that are applied to the surface, e.g., skin, of patient 16. Electrodes 18 may be electrodes pads, which may include an adhesive backing for attachment to the surface of patient 16, as is known in the art. Electrodes 18 are coupled to defibrillator 12 by respective leads or cables 20A and 20B (collectively “cables 20”). Although illustrated in FIG. 1 as coupled to two electrodes 18, external defibrillator 12 may be coupled to any number of electrodes 18, which may be incorporated into common electrode pads, and may share common cables 20. External defibrillator 12 may additionally or alternatively be coupled to patient 16 by sensors (not shown in FIG. 1), such as blood oxygen saturation or noninvasive blood pressure sensors.

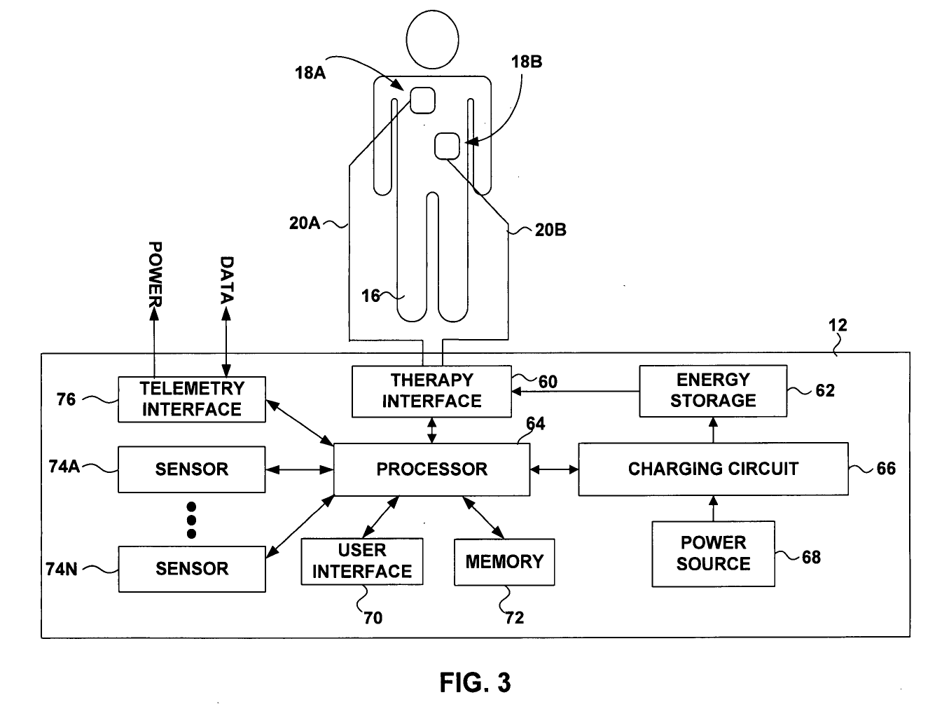

External defibrillator 12 detects electrical activity of the heart 22 of patient 16 via electrodes 18, and may deliver electrical stimulation to heart 22 via electrodes 18. For example, defibrillator 12 may deliver one or more defibrillation pulses to patient 16 via electrodes 18, as will be described in greater detail below with reference to FIG. 3. As shown in FIG. 1, defibrillator 12 may include a display 24, and may provide instructions in the form of prompts and other information to a user via the display. External defibrillator 12 may, for example, display an electrocardiogram generated based on the electrical activity detected by electrodes 18 via display 24. In some embodiments, defibrillator 12 may be coupled to additional sensors for sensing other physiological parameters of patient 16, such as blood pressure and oxygen saturation, and may display current or average values for the additional parameters via display 24.

In the illustrated example, IMD 14 is a multi-chamber cardiac pacemaker coupled to leads 26A-26C (collectively “leads 26”) that extend to selected positions within heart 22. As an alternative or in addition to pacing pulses, IMD 14 may deliver cardioversion and/or defibrillation pulses to heart 22 via leads. In other words, IMD 14 may be an implantable cardioverter defibrillator (ICD), as is known in the art. Further, IMD 14 may sense electrical activity of heart 22 via leads 26.

Leads 26 may include any of a variety of types of electrodes (not shown) known in the art for use in sensing cardiac electrical activity and delivering these types of stimulation to heart 22. The number and positions of leads 26 depicted in FIG. 1 are merely exemplary. Further, the invention is not limited to systems 10 in which an IMD is a pacemaker. IMD 14 may be any type of IMD that senses one or more physiological parameters of patient 16 and/or delivers one or more therapies to the patient. For example, IMD 14 may be an implantable neurostimulator, muscle stimulator, gastrointestinal stimulator, an implantable pump, or an implantable monitor such as an implantable loop recorder.

External defibrillator 12 wirelessly communicates with IMD 14, i.e., without being coupled to the IMD by a wire or other electrical conductor. In some embodiments, external defibrillator 12 wirelessly communication with IMD 14 via telemetry circuitry of the IMD, which may also be used by dedicated programming devices to communicate with the IMD. Dedicated programming devices may communicate with IMD via its telemetry circuitry to program or reprogram the operating parameters of the IMD, or to retrieve information stored or collected by the IMD, as is known in the art. Like dedicated programming devices, external defibrillator 12 may include corresponding telemetry circuitry to facilitate communication with IMD 14 via its telemetry circuitry. The telemetry circuitry of external defibrillator 12 and IMD 14 may include transceivers and antennas for communication via a radio-frequency (RF) communication medium, e.g., for communication via RF telemetry.

In the example illustrated by FIG. 1, external defibrillator 12 is coupled to a telemetry head 28 by a cable 30. Telemetry head 28 may include an antenna, and may be placed proximate to, e.g., over, IMD 14 by a user of defibrillator 12 to enable the external defibrillator to detect and communicate with the IMD. Defibrillator 12 may be removably coupled to telemetry head 28 by cable 30. In some embodiments, telemetry head 28 may be integral with a housing of external defibrillator 12, or incorporated into one of electrodes 18 and coupled to the external defibrillator by a lead 20.

In other embodiments, the telemetry circuitry and antennae of external defibrillator 12 and IMD 14 may support a signal strength, other signal characteristics, and communication protocol that allow RF telemetry communication between the external defibrillator and IMD at relatively greater distances. In such embodiments, one or more antennae of external defibrillator 12 may be housed within the defibrillator, i.e., external defibrillator 12 need not be coupled to telemetry head 28 to communicate with the IMD, and defibrillator 12 may detect and communicate with IMD when brought into proximity with the IMD.

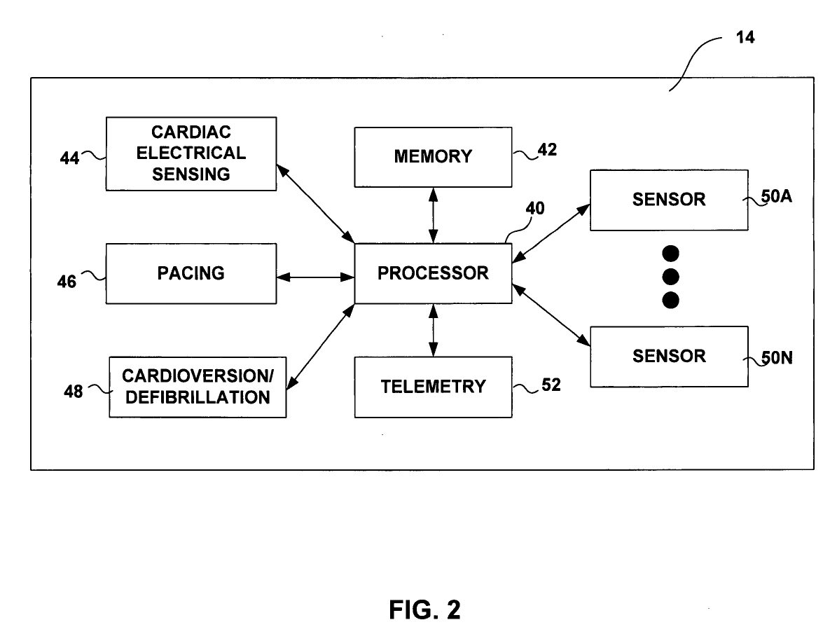

FIG. 2 is a block diagram further illustrating IMD 14. In the illustrated embodiment, IMD 14 is a cardiac pacemaker that is capable of delivering cardioversion and/or defibrillation pulses to patient 16, e.g., an ICD. However, as discussed above, IMD 14 need not include cardioversion or defibrillation capabilities, and need not be a cardiac pacemaker. The configuration of IMD 14 illustrated in FIG. 2 is merely exemplary.

IMD 14 includes a processor 40. Processor 40 executes program instructions stored in a memory 42, which control processor 40 to perform the functions ascribed to processor 40 and IMD 14 herein. Processor 40 may include any one or more of a microprocessor, digital signal processor (DSP), application specific integrated circuit (ASIC), field-programmable gate array (FPGA), or other digital logic circuitry. Memory 42 may include, for example, any one or more of a random access memory (RAM), read only memory (ROM), electronically erasable programmable ROM (EEPROM), or flash memory.

IMD 14 includes cardiac electrical sensing circuitry 44 coupled to the electrodes carried by leads 26 to sense electrical activity within heart 22. Cardiac electrical sensing circuitry 44 may include amplifiers, such as automatic gain controlled amplifier providing an adjustable sensing threshold. Such amplifiers may be used to detect the occurrence of R-waves, P-waves, or other morphological features within the signals detected by the electrodes, as is known in the art. For example, such amplifiers may output a signal to processor 40 when the amplitude of a signal detected by the electrodes exceeds a threshold associated with the morphological feature of interest.

Cardiac electrical sensing circuitry 44 may also include an amplifier, filter and an analog-to-digital converter to provide digital versions of the signals sensed by the electrodes carried by lead 26 to processor 40, e.g., to provide a digital electrocardiogram (ECG) signal to processor 40. Processor 40 may process the digital ECG to, for example, detect and classify arrhythmias of heart 22. Processor 40 may also store samples of the ECG in memory 42. Further, processor 40 may store various information regarding the rate or performance of heart 22 in memory determined based on the signals received from the amplifiers of cardiac electrical sensing circuitry 44 or analysis of the ECG received from the cardiac electrical sensing circuitry.

IMD 14 also includes pacing circuitry 46 that delivers pacing pulses to heart 22 via the electrodes carried by leads 26. Pacing circuitry 46 may include capacitors and switches for the storage and delivery of energy as a pacing pulse. Processor 40 controls the storage of energy and delivery of pacing pulses by pacing circuitry 46 by, for example, controlling the configuration of the switches.

Processor 40 may control pacing circuitry 46 to deliver pacing pulses according to any of a variety of known pacing modes, such as DDD, DDI, VVI, VOO and VVT modes. In some embodiments, processor 40 controls pacing circuitry 46 to deliver pacing pulses according to any of a variety of known rate-responsive pacing modes, including, but not limited to, DDDR, DDIR, VVIR, VOOR and VVTR. In some embodiments, processor may control pacing circuitry 46 to deliver pacing pulses to the ventricles of heart 22 at different times to provide cardiac resynchronization therapy.

Further, processor 40 may control pacing circuitry 46 to provide post extra-systolic potentiation (PESP) pacing by delivering of an extra-systolic pacing pulse to a chamber of heart 22 a relatively short interval after a paced or intrinsic depolarization of that chamber, e.g., within the relative refractory period after the first paced or spontaneous depolarization. Delivery of an extra-systolic pacing pulse may result in a second electrical depolarization of the chamber without an attendant myocardial contraction, which may effectively prolong the refractory period after the mechanical contraction of the chamber caused by the first paced or intrinsic depolarization.

The prolonged refractory period caused by PESP effectively slows the heart rate from its spontaneous rhythm, allowing a greater time for filling of the chamber. Further, PESP may cause a potentiation of contractile force of the chamber during the heart cycle that the extra-systolic pulse is applied. Increased filling and contractile force potentiation can lead to increased cardiac output, particularly when PESP is delivered to one or more of the ventricles of the heart.

Processor 40 may maintain programmable digital counters, and may control delivery of pacing pulses based on expiration of the programmable digital counters. Processor 40 may use the programmable counters to time various intervals associated with a selected mode of pacing. For example, processor 40 may use the digital counters to time the various atrial, ventricular, atrioventricular, interventricular, or extra-systolic intervals associated with the various modes of pacing discussed above. Processor 40 may set or reset the counters based delivery of pacing pulses by pacing circuitry 46 or detection of intrinsic R-waves or P-waves via cardiac electrical sensing circuitry 44.

In some embodiments, processor 40 detects arrhythmias, e.g., ventricular and/or atrial tachycardias or fibrillations of heart 22, using tachycardia and fibrillation detection techniques and algorithms known in the art. For example, processor 40 may detect the presence of a ventricular or atrial tachycardia or fibrillation by detecting sustained series of short R-R or P-P intervals of an average rate indicative of tachycardia, or an unbroken series of short R-R or P-P intervals, based on signals output by cardiac electrical sensing circuitry 44. Processor 40 may control pacing circuitry 46 to deliver one or more anti-tachycardia pacing (ATP) therapies to heart 22 in response to detection of an arrhythmia. IMD 14 may also include cardioversion/defibrillation circuitry 48, which processor 40 may control to deliver cardioversion or defibrillation pulses to heart 22 response to detection of an arrhythmia. Circuitry 48 may include energy storage circuits such as capacitors, and switches for coupling the storage circuits to electrodes carried by leads 26.

As illustrated in by the example of FIG. 2, in addition to cardiac electrical sensing circuitry 44, IMD 14 may include additional sensors 50A-50N (collectively “sensors 50”) that output signals as a function of other physiological parameters. In some embodiments, one or more sensors 50 may be coupled to IMD 14 via leads 26. IMD 14 may include circuitry that conditions the signals generated by sensors 50 such that they may be analyzed by processor 40. For example, IMD 14 may include one or more analog to digital converters to convert analog signals generated by sensors 50 into digital signals usable by processor 40, as well as suitable filter and amplifier circuitry. As examples, IMD 14 may include known sensors and circuitry to detect patient activity, posture, respiration, thoracic impedance, blood pressure, intracardiac pressure, blood flow, temperature, pH, blood oxygen saturation, or the partial pressure of oxygen or carbon dioxide in the blood of patient 16. Processor 40 may use the signals output by sensors 50 to, for example, adjust the aggressiveness of rate responsive pacing delivered to heart 22.

Further, as discussed above, IMD 14 includes telemetry circuitry 52. Telemetry circuitry may include a transceiver and one or more antennae for communicating with programming devices and defibrillator 12 via an RF medium. Telemetry circuitry 52 may also include circuitry that conditions signals between the transceiver and processor 40, such as one or more analog to digital and digital to analog converters, as well as suitable filter and amplifier circuitry.

FIG. 3 is a block diagram further illustrating external defibrillator 12. In FIG. 3, external defibrillator 12 is shown coupled to patient 16 by electrodes 18 and corresponding cables 20, as described above. In a typical application, a therapy interface 60 of defibrillator 12 includes a receptacle, and cables 20 plug into the receptacle.

Therapy interface 60 includes a switch (not shown in FIG. 3) that, when activated, couples an energy storage circuit 62 to electrodes 18. Energy storage circuit 62 stores energy to be delivered to patient 16 in the form of a defibrillation pulse. The switch may be of conventional design and may be formed, for example, of electrically operated relays. Alternatively, the switch may comprise an arrangement of solid-state devices such as silicon-controlled rectifiers or insulated gate bipolar transistors.

Energy storage circuit 62 includes components, such as one or more capacitors, that store the energy to be delivered to patient 16 via electrodes 18. Before a defibrillation pulse may be delivered to patient 16, energy storage circuit 62 must be charged. A processor 64 directs a charging circuit 66 to charge energy storage circuit 62 to a high voltage level. Charging circuit 66 comprises, for example, a flyback charger that transfers energy from a power source 68 to energy storage circuit 62.

As indicated above, external defibrillator 12 may be a manual defibrillator or an AED. Where external defibrillator 12 is a manual defibrillator, a user of defibrillator 12 may select an energy level for each defibrillation pulse delivered to patient 16. Processor 64 may receive the selection made by the user via a user interface 70, which may include input devices, such as a keypad and various buttons or dials, and output devices, such as various indicator lights, display 24 (FIG. 1), and a speaker. Display 24 may include a cathode ray tube (CRT), light emitting diode (LED), or liquid crystal display (LCD) screen.

Where defibrillator 12 is an AED, processor 64 may select an energy level. For example, processor 64 may select an energy level from a preprogrammed progression of energy levels stored in a memory 72 based on the number of defibrillation pulses already delivered to patient 16. In some manual defibrillator embodiments, processor 64 may select an energy level, e.g., based on a preprogrammed progression, to recommend to a user via user interface 70.

In either case, when the energy stored in energy storage circuit 62 reaches the desired energy level, processor 64 controls user interface 70 to provide an indication to the user that defibrillator 12 is ready to deliver a defibrillation pulse to patient 16, such as an indicator light or a voice prompt. The defibrillation pulse may be delivered manually or automatically. Where the defibrillation pulse is delivered manually, the user may direct processor 64 to deliver the defibrillation pulse via user interface 70 by, for example pressing a button. In either case, processor 64 activates the switches of interface 60 to electrically connect energy storage circuit 62 to electrodes 18, and thereby deliver the defibrillation pulse to patient 16. Therapy interface 60, energy storage circuitry 62 and charging circuit 66 are examples of therapy delivery circuitry that deliver therapy to patient 16 under control of processor 64.

Processor 64 may modulate the defibrillation pulse delivered to patient 16. Processor 64 may, for example, control the switches of interface 60 to regulate the shape and width of the pulse. Processor 64 may control the switches to modulate the pulse to, for example, provide a multiphasic pulse, such as a biphasic truncated exponential pulse, as is known in the art.

Processor 64 may perform other functions as well, such as monitoring electrical activity of the heart of patient 16 sensed via electrodes 18. Therapy interface 60 may include circuitry for sensing the electrical activity of the heart via electrodes 18. Processor 64 may determine whether heart 22 of patient 16 is fibrillating based upon the sensed electrical activity in order to determine whether a defibrillation pulse should be delivered to patient 16. Where a defibrillation pulse has already been delivered, processor 64 may evaluate the efficacy of the delivered defibrillation pulse by determining if heart 22 is still fibrillating in order to determine whether an additional defibrillation pulse is warranted. Processor 64 may automatically deliver defibrillation pulses based on these determinations, or may advise the caregiver of these determinations via user interface 70. Processor 64 may display an electrocardiogram (ECG) that reflects the sensed electrical activity via user interface 70, e.g., via display 24 (FIG. 1).

Processor 64 may store an indication of the time of delivery of each defibrillation pulse delivered to patient 16 as medical event information within memory 72 for patient 16. Processor 64 may also store the energy level of each pulse and other characteristics of each pulse, such as the width, amplitude, or shape, as medical event information for patient 16. Processor 64 may also store a digital representation of the ECG, or a heart rate over time determined based on the electrical activity of the heart of patient 34 detected via electrodes 18 within memory 72 as medical event information for patient 16. Further, processor 64 may control delivery of other types of therapy to patient 16 via electrodes 18, such as cardioversion or pacing therapy, and store information describing the times that such therapies were delivered and parameters of such therapies, such as cardioversion pulse energy levels and pacing rates, as medical event information for patient 16.

User interface 70 may include a microphone (not shown) that detects sounds in the vicinity of defibrillator 12. Processor 64 may receive signals from the microphone and store an audio recording that includes these signals within memory 72 as medical event information for patient 34. The audio recording may include verbal notations of a user of defibrillator 12, or conversations between the user and patient 16. Additionally, the user may mark the time of the occurrence of various events, such as the delivery of drugs or the administration of cardiopulmonary resuscitation (CPR), during the treatment of patient 16 by, for example, pressing a key or button of user interface 70 at the time when the event occurred. These event markers may also be included within the medical event information stored in memory 72 for patient 16. Additionally or alternatively, processor 64 may also detect on-going adjunct therapies such as CPR or ventilation from any signal, e.g., electrical, impedance, optical, or magnetic, and store an indication of the occurrence of such adjunct therapies as medical event information for patient 16 within memory 72.

Where external defibrillator 12 is more fully featured, e.g., a manual paramedic or hospital defibrillator, defibrillator 12 may also include additional sensors 74A-74N (collectively “sensors 74”) coupled to processor 64, such as sensors to measure blood oxygen saturation, blood pressure, respiration, and the amount of oxygen or carbon dioxide in the air inhaled or exhaled by patient 16. Sensors 74 may be included within or coupled to external defibrillator 12. External defibrillator 12 may include circuitry that conditions the signals generated by sensors 74 such that they may be analyzed by processor 64, such as one or more analog to digital converters to, as suitable filter and amplifier circuitry.

Processor 64 may also store the signals generated by these sensors within memory 72 as medical event information for patient 16. In other words, as examples, processor 64 may also store any of a capnograph, a plethysmograph, a blood oxygen saturation over time, a blood pressure over time, a pulse rate over time determined based on measured blood pressure, end tidal carbon dioxide measurements, and/or measurements of the fraction of carbon dioxide in air inspired or expired within memory 72 as medical event information for patient 16. Processor 64 may also receive other information collected by a user during treatment of patient 16, such as a location of treatment or time of death, and store such information as medical event information for the patient. Processor 64 may begin to store medical event information 32 when defibrillator 12 is powered on to respond to a medical emergency involving patient 16.

Processor 64 may, for example, include one or more of a microprocessor, DSP, ASIC, FPGA, or other logic circuitry. Memory 72 may include program instructions that cause processor 64 to perform the functions attributed to processor 64 and defibrillator 12 herein. Accordingly, the invention also contemplates computer-readable media storing instructions to cause processor 64 to provide the functionality described herein. Memory 72 may include any of a variety of solid state, magnetic or optical media, such as RAM, ROM, CD-ROM, magnetic disk, EEPROM, or flash memory.

In the example illustrated by FIG. 3, external defibrillator 12 includes a telemetry interface 76. Telemetry interface 76 may include a port or other physical interface to receive cable 30 that is coupled to telemetry head 28 (FIG. 1), and to electrically couple the circuitry within defibrillator 12 to the circuitry within telemetry head 28 via cable 30. Processor 64 communicates with IMD 14 via telemetry interface 76 and telemetry head 28.

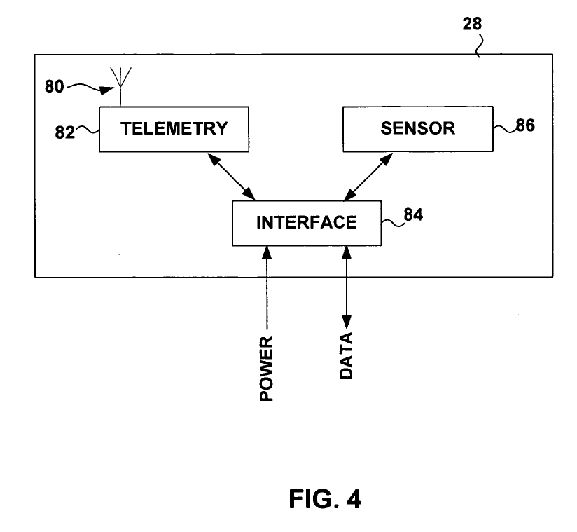

In some embodiments, as illustrated in FIG. 3, interface 76 may convey data between processor 64 and telemetry head 28, as well as provide power from defibrillator 12 to power the circuitry within telemetry head 28. In some embodiments, as will be described below with reference to FIG. 4, telemetry head 28 may incorporate telemetry circuitry including a transceiver and one or more analog to digital and digital to analog converters, in addition to one or more antennae for communication with IMD 14. In such embodiments, telemetry interface 74 may include any of a variety of known digital data interfaces, such as a universal serial bus (USB) port.

In other embodiments, external defibrillator 12 may include the telemetry circuitry, and telemetry head 28 may include only one or more antennae for communication with IMD 14. Further, in still other embodiments, defibrillator 12 may include both telemetry circuitry and antennae for communication with IMD 14. In such embodiments, defibrillator 12 need not be coupled to telemetry head 28 for in order to communicate with IMD 14.

FIG. 4 is a block diagram further illustrating telemetry head 28 according to an embodiment of the invention. In the illustrated example, telemetry head 28 includes an antenna 80 coupled to telemetry circuitry 82. Telemetry circuitry 82 may include a transceiver for wireless communication with IMD 14 via antenna 80 and an RF medium. Telemetry circuitry 82 may also include various circuitry for conditioning signal transmitted or received via antenna 80, such as analog to digital and digital to analog converters, and appropriate amplifiers or filters.

An interface 84 of telemetry head 28 interfaces with telemetry interface 76 of external defibrillator 12. Interface 84 may include a plug or other physical interface on cable 30 that may be used to removeably couple telemetry head 28 to defibrillator 12, and which electrically couples the circuitry within telemetry head 28 to circuitry within defibrillator 12 via telemetry interface 76. As illustrated in FIG. 4, interface 84 may convey data between telemetry circuitry 82 and external defibrillator 12, and may receive power from defibrillator 12 for distribution to the various components of telemetry head 28. Interface 84 may include any of a variety of known digital data interfaces, such as a universal serial bus (USB) plug.

As illustrated in FIG. 4, telemetry head 28 may additionally include one or more sensors 86. Because telemetry head 28 may be positioned on the surface of patient 16, sensors 86 located on or within telemetry head 28 may be able to sense a variety of physiological parameters of patient 16. For example, a sensor 86 may be an oxygen saturation, temperature, blood flow, pulse rate, or heart sound sensors. Telemetry head 28 may include circuitry that conditions the signals generated by sensor 86 such that they may be transmitted to external defibrillator 12 via interface 84 in digital form, such as one or more analog to digital converters to, as suitable filter and amplifier circuitry. By incorporating sensor 86 into telemetry head 28, external defibrillator 12 may be able to sense a physiological parameter of patient 16 that it would not otherwise be able to sense. Further, to the extent that defibrillator 12 may have separately included or been coupled to a sensor that sensed the same physiological parameter as sensor 86, incorporation of sensor 86 into telemetry head 28 may reduce the number of separate sensor apparatuses and associated cables coupled to defibrillator 12 while in use, reducing the potential for obstruction or confusion when a user of the external defibrillator is treating patient 16.

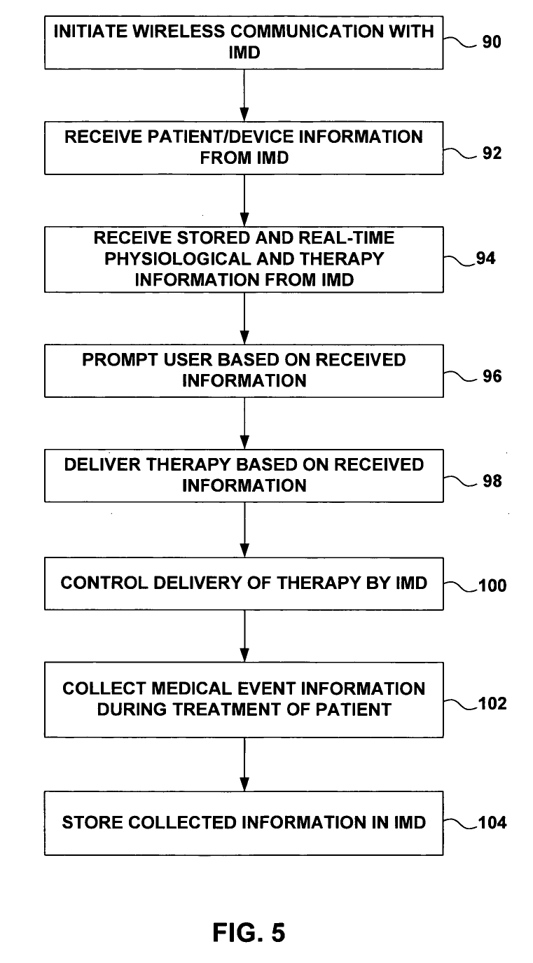

FIG. 5 is a flow diagram illustrating an example method that may be employed by external defibrillator 12 that communicates with IMD 14 during treatment of patient 16. According to the example method, external defibrillator 12 initiates wireless communication, e.g., initiates a telemetry session, with IMD 14 (90). For example, when a user of external defibrillator 12 arrives at the scene of a medical emergency involving patient 16 with the defibrillator, the user may couple cable 30 to external defibrillator 12, and place telemetry head 28 on the chest or abdomen of patient 16. Processor 64 of the external defibrillator may detect coupling of cable 30 to telemetry interface 76, and may begin attempting to contact IMD 14 to initiate the telemetry session in response to detecting the coupling. In other embodiments, such as embodiments in which external defibrillator 12 houses telemetry circuitry and antennae for communication with IMD 14, processor 64 may begin attempting to contact IMD 14 at another time, such as when external defibrillator 12 is powered on. In still other embodiments, processor 64 may begin attempting to contact IMD 14 upon receipt of a command from the user via user interface 70 of external defibrillator 12.

IMD 14 may store a variety of information regarding patient 16 and IMD 14 itself within memory 42, and defibrillator 12 may retrieve this information from IMD 14 during the telemetry session (92). For example, memory 42 may store demographic information for patient 16, such as name, height, weight, sex, age, date of birth, and the like. Further, memory 42 may store treatment alerts for patient 16, such as medications taken by the patient, allergies of the patient, or a do not resuscitate (DNR) order for the patient. Memory 42 may store information describing the type of IMD 14, its lead configuration, and current programmed parameters, such as a current pacing mode. Memory 42 may also store information identifying the implant location of IMD 14.

When processor 64 of external defibrillator 12 receives such information from IMD 14, processor 64 may store the information in memory 72 as medical event information for patient 16. Such information may then be included in a report of the treatment of patient 16, e.g., a “run” report, along with other medical event information collected by external defibrillator 12 as discussed above with reference to FIG. 3. Paramedics, first responders, or other users of external defibrillator 12 h may be required to prepare such run reports by an emergency medical service or other regulating authority. Because external defibrillator 12 may retrieve such patient and device information from IMD 14 and include the information within the medical event information for patient 16 automatically, a user of the external defibrillator may not be required to take time to collect such information from patient 16, family members, or bystanders, and enter the information into external defibrillator 12 manually via user interface 70 of the defibrillator. Consequently, the user's time and attention may remain focused on treating patient 16.

IMD 14 may also store physiological and therapy information within memory 42. External defibrillator 12 may retrieve this stored information from IMD 14, and may also receive real-time values for one or more physiological parameters and real-time indications therapies delivered or scheduled for delivery by the IMD from the IMD (94). For example, external defibrillator 12 may receive ECG samples recorded and stored by IMD 14, and may receive a real-time ECG sensed by IMD 14 via leads 26. External defibrillator 12 may store any or all of the past or real-time information received from IMD 14 within memory 72.

Further, external defibrillator 12 may receive heart rate data stored by IMD 14, including average values or other statistical summaries of the heart rate of patient 16 over time. External defibrillator 12 may also receive current heart rate values, or current average heart rate value, e.g., averaged over a relatively short period of time such as a minute, from the IMD. External defibrillator 12 may also receive stored or real-time values for other physiological parameters that may be detected by IMD 14 as discussed above, such as blood pressure and blood flow.

In some embodiments, IMD 14 may determine the time at which a medical emergency involving patient 16 began, e.g., when the patient first experienced a fibrillation or sudden cardiac arrest (SCA). In embodiments in which IMD 14 is a cardiac pacemaker, for example, the IMD may detect onset of fibrillation or SCA through analysis of an electrocardiogram of the patient. In some embodiments, sensors 50 of IMD 14 may include one or more DC accelerometers, mercury switches, or gyroscopes to detect the posture of patient 16, and may IMD 14 may detect that patient 16 has collapsed as a result of a medical emergency, such as fibrillation or SCA. External defibrillator 12 may receive information indicating the time of onset of the medical emergency from the IMD.

Additionally, external defibrillator 12 may receive information stored by IMD 14 indicating when the IMD has delivered therapies to patient 16. For example, defibrillator 12 may receive information stored by IMD 14 indicating the time and energy level of defibrillation pulses delivered to patient 16 by the IMD. Further, IMD 14 may notify external defibrillator 12 that the IMD is scheduled to or has otherwise decided to deliver a therapy to patient 16. For example, IMD 14 may indicate to external defibrillator 12 that it has detected a shockable arrhythmia, and may indicate an energy level and delivery time of a defibrillation pulse that it will deliver to heart 22 in response to detecting the arrhythmia. Processor 64 of external defibrillator 12 may store the physiological and therapy information received from IMD 14 in memory 72 as medical event information for the patient.

Processor 64 of external defibrillator 12 may provide prompts to a user via user interface 70, e.g., via a speaker and/or display 24, based on the information received from IMD 14 (96). In some embodiments, providing prompts based on the information received from IMD comprises modifying programmed prompts that may have otherwise been provided to a user of defibrillator 12 in the absence of communication with IMD 14. For example, memory 72 of external defibrillator 12 may store graphical or audible prompts provided to a user by processor 64 that indicate locations for the user place electrodes 18 on patient 16. If an implant location for IMD 14 received from the IMD indicates that the IMD is implanted proximate one of the default electrode locations, processor 64 may provide a modified prompt to direct the user place electrodes 18 at alternative locations. By placing electrodes 18 at locations some distance form the implant location of IMD 14, interference between external defibrillator 12 and IMD 14 may be reduced. Interference between external defibrillator 12 and IMD 14 may include electromagnetic interference, which may degrade the signals generated by respective sensors 50, 74.

As another example, processor 64 may prompt a user of external defibrillator 12 with patient treatment alert information received from IMD 14. For example, processor 64 may provide prompts to the user indicating allergies, potential drug interactions, or a DNR order for patient 16. Because patient treatment alert information may impact treatment decisions made by a user of external defibrillator 12, processor 64 may use bold or flashing text, flashing lights, audible alerts, or the like to draw the attention of the user to the presence of one or more patient treatment alerts.

Additionally, processor 64 prompts user with a time of onset of the current medical emergency, or a time elapsed since onset of the medical emergency, based on the time of onset information received from IMD 14. The efficacy of therapies that could be delivered patient may vary based on the amount of time elapsed since onset of the medical emergency, e.g., amount of time in fibrillation or SCA. Consequently, a user of external defibrillator 12 may provide different therapies to patient 16 based on the time of onset or amount of time elapsed indicated by external defibrillator 12 based on information received from IMD 14. For example, a user of external defibrillator 12 may elect to deliver defibrillation pulses to patient 16 if the patient has been in SCA or fibrillation for less than five minutes, and elect to perform CPR on the patient if the patient has been in SCA or fibrillation for greater than five minutes. In some embodiments, external defibrillator 12 may prompt the user to provide a particular therapy or type of monitoring based on the onset or elapsed time information received from IMD.

Further, if the received information indicates that IMD 14 is scheduled to deliver a therapy to patient 16, processor 64 may provide a prompt notifying the user of the upcoming delivery of therapy. For example, IMD 14 may identify a shockable arrhythmia of heart 22, and transmit an indication to external defibrillator 12 that IMD 14 will deliver a defibrillation pulse to the heart. Processor 64 may direct the user to avoid contact with patient, e.g., stop CPR, for a period of time to avoid receiving a portion of the energy of the defibrillation pulse delivered by IMD 14, which may cause discomfort or injury to the user.

Processor 64 may also display some or all of the information received from IMD 14 via display 24. For example, processor 64 may receive and display the name of patient 16 as stored by IMD 14, allowing a user of external defibrillator 12 to address the patient by name without having to ask the patient, family members, or other bystanders.

Further, processor 64 may display real-time values of physiological parameters sensed by IMD 14, such as a real-time ECG sensed by IMD 14 via leads 26, via display. Through communication with IMD 14, external defibrillator 12 may be able to display values of physiological parameters that may not have otherwise been able to be sensed by defibrillator 12. Processor 64 may provide prompts based on some of these values. For example, processor 64 may provide audio or textual prompts regarding the efficacy of CPR provided by a user of external defibrillator 12, e.g., instruction to apply more or less forceful chest compressions, based on blood pressure or blood flow values measured by IMD 14.

An ECG detected by IMD 14 via leads 26 may be of a higher quality than an ECG detected by external defibrillator 12 via electrodes 18. For example, an ECG detected by IMD 14 may be less likely to include motion artifacts caused by CPR chest compressions than an ECG detected by the external defibrillator. Consequently, where available from IMD 14, processor 64 of the external defibrillator may display a real-time ECG received from IMD 14. In some embodiments, the processor may select either the ECG detected by the external defibrillator or received from the IMD based on a criteria related to the quality of the ECGs, such as noise or impedance. For example, the processor may select the IMD ECG when available unless signal to noise ratio of the external ECG, i.e., the ECG detected by the defibrillator, is above a threshold value.

Processor 64 may also display information indicating therapies delivered to patient 16 by IMD 14 via display 22. If the displayed information indicates that the IMD has already delivered therapy to patient 16 in response to the current medical emergency, the user may consider such information and thereby avoid delivering redundant therapies to patient 16. For example, the displayed information may indicate energy levels of defibrillation pulses delivered to patient by IMD 14, and the user may select an energy level for a defibrillation pulse to delivered by external defibrillator 12 that is adjusted based on the energy levels of the defibrillation pulses delivered by the IMD. For example, the user may select an energy level for a defibrillation pulse to delivered by external defibrillator 12 that is greater than the energy levels of the defibrillation pulses delivered by the IMD if the pulse delivered by the IMD failed to defibrillate heart 22.

External defibrillator 12 may also deliver therapy to patient 16 based on the information received from IMD 14 (98). For example, in embodiments in which processor 64 selects an energy level for a defibrillation pulse to be delivered to patient 16 by external defibrillator 12, processor 64 may select the energy level based on the information. The information received from IMD 14 may indicate an energy level of a defibrillation pulse delivered to patient 16 by IMD 14, and processor 64 may select an energy level for a defibrillation pulse to be delivered by external defibrillator 12 based on the indicated energy level. Processor 64 may select a higher energy level to avoid delivering a redundant defibrillation pulse which may have already proven ineffective at ending fibrillation of heart 22.

As another example, in embodiments in which processor 64 analyzes an ECG to determine whether to deliver therapy, e.g., a defibrillation pulse, to patient 16, processor 64 may analyze a real-time ECG received from IMD 14. As discussed above, the ECG received from IMD 14 may be of a higher quality, e.g., less susceptible to motion artifacts from CPR chest compressions, than an ECG detected via electrodes 18. Consequently, by using an ECG received from IMD 14, processor 64 may be able to more accurately determine whether therapy should be delivered to patient 16. Additionally, as discussed above, processor 64 may select one of the IMD and external ECG for analysis based on a criterion related to the quality of at least one of the ECGs.

Further, in some embodiments, IMD 14 may use different algorithms to determine whether to deliver therapy to patient 16 then are available to processor 64, and processor 64 may deliver therapy based on a therapy delivery decision received from IMD 14. For example, IMD 14 may apply arrhythmia detection algorithms to the rhythm of heart 22 that distinguish between ventricular and supra-ventricular arrhythmias. IMD 14 may decide that a defibrillation pulse should be delivered in response to detection of a ventricular arrhythmia, and that a defibrillation pulse should not be delivered in response to detection of a supra-ventricular arrhythmia. Processor 64 may control delivery of a defibrillation pulse to patient 16 based on a defibrillation pulse delivery decision received from IMD 14. In this manner, external defibrillator 12 may, for example, avoid delivering a defibrillation pulse to treat a supra-ventricular arrhythmia. In some embodiments, a user may override a decision by processor 64 not to deliver therapy based on information received from IMD 14, and direct defibrillator 12 to deliver therapy.

Additionally, processor 64 may control delivery of therapy by external defibrillator 12, e.g., control charging circuit 66 and therapy delivery interface 60, based on onset or elapsed time information received from IMD 14. For example, processor 64 may select a therapy, such as defibrillation, cardioversion or pacing, or the energy levels for such therapy, based on the time. Processor 64 may alternatively suspend delivery of therapy by external defibrillator 12 based on the time information.

Processor 64 of external defibrillator 12 may also control delivery of therapy by IMD 14 (100). For example, processor 64 may suspend delivery of therapy by IMD 14 during treatment of patient 16 with external defibrillator 12. By suspending delivery of therapy by IMD 14, external defibrillator 12 may avoid interference between therapies delivered by IMD 14 and defibrillator 12.

As another example, processor 64 may change a therapy delivery mode of IMD 14. For example, after defibrillation by external defibrillator 12, some patients may benefit from pacing in a different mode than the mode in which IMD 14 had been programmed. Processor 64 may change the mode of IMD 14 by, for example, changing IMD 14 from single to dual chamber pacing or from demand to non-demand pacing, or by changing a pacing rate or the aggressiveness of rate responsive pacing.

Further, the hearts of some patients are left in a state of pulseless electrical activity after being defibrillated. Such patients may benefit from delivery of post extra-systolic potentiation (PESP) pacing, which may increase the cardiac output of their heart. If IMD 14 is capable of delivering post extra-systolic pacing pulses, processor 64 may direct IMD 14 to do so after heart 22 has been defibrillated. In some embodiments, processor 64 may direct IMD 14 to delivery other therapies provided by the IMD that may not be available from the external defibrillator, such as cardioverion or anti-tachycardia pacing therapies.

Additionally, processor 64 may direct IMD 14 to deliver therapy that is coordinated with therapy delivered by defibrillator 12. For example, processor 64 may direct IMD 14 to deliver a defibrillation pulse synchronized with, or with some other temporal relationship to, a defibrillation pulse delivered by defibrillator 12. Delivery of defibrillation pulses by both IMD 14 and external defibrillator 12 may be more efficacious than delivery of defibrillation pulses by either the external defibrillator or the IMD alone.

As another example, external defibrillator 12 may include pacing circuitry for delivery of pacing pulses to heart 22 of patient 16 via electrodes 18. To the extent IMD 14 is not capable of delivering post extra-systolic pacing pulses, processor 64 may control the pacing circuitry to deliver pacing pulses an extra-systolic interval after delivery of a pacing pulse by IMD 14, or an intrinsic depolarization of heart 22. Processor 64 of external defibrillator 12 may interrogate IMD 14 to identify the therapies sensing capabilities provided by the IMD. Processor 64 may control the IMD to deliver a therapy alone, or in coordination with the external defibrillator, based on this capability information retrieved from the IMD.

As described above, processor 64 collects medical event information during treatment of patient 16 with external defibrillator 12, and stores the medical event information within memory 72 of the external defibrillator (102). Processor 64 may also store the medical event information into IMD 14, e.g., within memory 42 of IMD 14 (104). In this manner, caregivers who subsequently treat patient 16 and have access to a programming device that communicates with IMD 14 may be able to retrieve the medical event information. In the absence of communication between IMD 14 and external defibrillator 12, such caregivers may not have had access or timely access to the medical event information, which may inform treatment decisions made by the caregivers, and may supplement the medical records maintained for patient 16 by the caregivers. In some embodiments, rather than a caregiver retrieving the information with a programming device, IMD 14 may transmit the medical event information to a computing device, computing network, or other data repository at, for example, a hospital. The medical event information may supplement the hospitals records for the patient, and may be available to caregivers throughout the hospital who may treat the patient.

Various embodiments of the invention have been described. However, one skilled in the art will appreciate that various modifications may be made to the described embodiment without departing from the scope of the claimed invention. For example, although wireless communication has been described herein primarily in the context of RF telemetry, the invention is not so limited. An external defibrillator and IMD according to the invention may include any of a variety of RF, optical, acoustic, or other transducers for wireless communication. Further, although described in the context of communication with an IMD, an external defibrillator according to the invention may communicate with other external medical devices that are associated with the patient, such as a wearable defibrillator or Holter monitor. These and other embodiments are within the scope of the following claims.

1. An external defibrillator comprising:
wireless communication circuitry to wirelessly communicate with an implantable medical device implanted within a patient;
therapy delivery circuitry; and
a processor to receive information from the implantable medical device via the wireless communication circuitry, and control the therapy delivery circuitry to deliver therapy to the patient based on the received information.

2. The external defibrillator of claim 1, wherein the processor selects an energy level for a defibrillation pulse to be delivered to the patient based on the received information.

3. The external defibrillator of claim 1, wherein the processor suspends delivery of therapy by the therapy delivery circuitry based on the received information.

4. The external defibrillator of claim 1, wherein the received information includes real-time values of a physiological parameter of the patient.

5. The external defibrillator of claim 1, wherein the received information includes a real-time electrocardiogram of the patient, and the processor analyzes the electrocardiogram to determine whether to control the therapy delivery circuit to deliver a defibrillation pulse to the patient.

6. The external defibrillator of claim 5, further comprising circuitry for sensing an external electrocardiogram, wherein the processor selects one of the external electrocardiogram and the electrocardiogram received from the implantable medical device for analysis to determine whether to control the therapy delivery circuit to deliver a defibrillation pulse to the patient based on a criteria related to quality of at least one of the electrocardiograms.

7. The external defibrillator of claim 1, wherein the received information indicates at least one of a time of onset of a medical emergency or an elapsed time of the medical emergency, and the processor controls delivery of therapy based on the time of onset or elapsed time.

8. The external defibrillator of claim 1, wherein the processor collects medical event information during treatment of the patient with the external defibrillator, and transmits the collected information to the implantable medical device via the telemetry circuitry for storage within the implantable medical device.

9. The external defibrillator of claim 1, wherein the processor controls delivery of therapy by the implantable medical device via the telemetry circuitry.

10. The external defibrillator of claim 9, wherein the implantable medical device comprises a pacemaker, and the processor of the external defibrillator modifies a pacing mode of the implantable medical device.

11. The external defibrillator of claim 9, wherein the implantable medical device comprises a defibrillator, and the processor of the external defibrillator controls delivery of a defibrillation pulse by the implantable medical device.

12. The external defibrillator of claim 11, wherein the processor controls the therapy delivery circuitry to deliver a defibrillation pulse at a selected time relative to the delivery of the defibrillation pulse by the implantable medical device.

13. The external defibrillator of claim 9, wherein the implantable medical device comprises a pacemaker, and the processor of the external defibrillator controls the implantable medical device to deliver post extra-systolic pacing.

14. The external defibrillator of claim 13, wherein the processor controls delivery of pacing pulses by the therapy delivery circuitry and the implantable medical device to deliver post extra-systolic pacing.

15. The external defibrillator of claim 9, wherein the processor controls the implantable medical device to deliver at least one of a cardioversion pulse or anti-tachycardia pacing.

16. An external defibrillator comprising:
wireless communication circuitry to wirelessly communicate with an implantable medical device implanted within a patient; and
a processor to receive real-time values of a physiological parameter of the patient from the implantable medical device via the wireless communication circuitry.

17. The external defibrillator of claim 16, further comprising a display, wherein the processor displays the received physiological parameter values via the display.

18. The external defibrillator of claim 17, wherein the processor displays the received physiological parameter values as a waveform via the display.

19. The external defibrillator of claim 18, wherein the received physiological parameter values comprise an electrocardiogram of the patient, and the processor displays the electrocardiogram via the display.

20. The external defibrillator of claim 16, wherein the received physiological parameter values comprise at least one of blood pressure or blood flow values, and the processor provides cardiopulmonary resuscitation instructions to a user based on the blood pressure or blood flow values.

21. A method comprising:
wirelessly communicating with an implantable medical device implanted within a patient via an external defibrillator; and
receiving real-time values of a physiological parameter of the patient from the implantable medical device at the external defibrillator via the wireless communication.

22. The method of claim 21, further comprising a displaying the received physiological parameter values.

23. The method of claim 22, wherein displaying the received physiological parameter values comprises displaying the received physiological parameter values as a waveform.

24. The method of claim 23, wherein receiving real-time values of a physiological parameter comprises receiving an electrocardiogram of the patient, and displaying the received physiological parameter values comprises displaying the electrocardiogram.

25. The method of claim 21, wherein receiving real-time values of a physiological parameter comprises receiving at least one of blood pressure or blood flow values, the method further comprising providing cardiopulmonary resuscitation instructions to a user of the external defibrillator based on the blood pressure or blood flow values.

26. An external defibrillator comprising:
wireless communication circuitry to wirelessly communicate with an implantable medical device implanted within a patient;
a user interface; and
a processor to receive information from the implantable medical device via the telemetry circuitry, and prompt a user of the external defibrillator via the user interface based on the information.

27. The external defibrillator of claim 26, wherein the processor modifies programmed prompts based on the received information.

28. The external defibrillator of claim 26, wherein the information indicates an implanted location of the implantable medical device, and the processor prompts the user to apply electrodes coupled to the external defibrillator to positions on the patient based on the implanted location.

29. The external defibrillator of claim 26, wherein the information comprises a patient treatment alert, and the processor prompts the user with the patient treatment alert.

30. The external defibrillator of claim 26, further comprising a memory, wherein processor stores medical event information in the memory during treatment of the patient, the information received from the implantable medical device comprises at least one of patient information and implantable medical device information, and the processor stores the information received from the implantable medical device as part of the medical event information for the patient.

31. The external defibrillator of claim 26, wherein the received information indicates at least one of a time of onset of a medical emergency or an elapsed time of the medical emergency, and the processor prompts the user according to the time of onset or elapsed time.

32. The external defibrillator of claim 31, wherein the processor provides the time of onset or elapsed time to the user.

33. The external defibrillator of claim 31, wherein the processor prompts a user to provide a selected one of a plurality of therapies based on the time of onset or elapsed time.

34. A method comprising:
wirelessly communicating with an implantable medical device implanted within a patient via an external defibrillator;
receiving information from the implantable medical device at the external defibrillator via the wireless communication; and
prompting a user of the external defibrillator based on the received information.

35. The method of claim 34, wherein prompting a user comprises modifying programmed prompts based on the received information.

36. The method of claim 34, wherein the information indicates an implanted location of the implantable medical device, and prompting a user comprises prompting the user to apply electrodes coupled to the external defibrillator to positions on the patient based on the implanted location.

37. The method of claim 34, wherein the information comprises a patient treatment alert, and prompting a user comprises prompting the user with the patient treatment alert.

38. The method of claim 34, wherein the information received from the implantable medical device comprises at least one of patient information and implantable medical device information, and the method further comprises:
storing medical event information during treatment of the patient; and
including the information received from the implantable medical device as part of the medical event information for the patient.

39. The method of claim 34, wherein the receiving information comprises receiving information that indicates at least one of a time of onset of a medical emergency or an elapsed time of the medical emergency from the implantable medical device, and prompting a user comprises prompting the user according to the time of onset or elapsed time.

40. The method of claim 39, wherein prompting a user according to the time of onset or elapsed time comprises providing the time of onset or elapsed time to the user.

41. The method of claim 39, wherein prompting a user comprises prompting the user to provide a selected one of a plurality of therapies based on the time of onset or elapsed time.
